(19) United States
(12) Patent Application Publication
(10) Pub. No.: US 2024/0425738 A1
(43) Pub. Date: Dec. 26, 2024

(54) FORMULATIONS WITH IMPROVED SPRAY CHARACTERISTICS

(71) Applicant: S. C. Johnson & Son, Inc., Racine, WI (US)

(72) Inventors: Randy Washington, Lake Villa, IL (US); Therese A. Rozek, Union Grove, WI (US); Shayle Blumberg, Lake Villa, IL (US)

(21) Appl. No.: 18/750,743

(22) Filed: Jun. 21, 2024

Related U.S. Application Data
(60) Provisional application No. 63/522,338, filed on Jun. 21, 2023.

(51) Int. Cl. C09K 3/30

(52) U.S. Cl. CPC C09K 3/30

(57) ABSTRACT

Disclosed herein are formulations with a solvent system that may be used in aerosol, compressed gas, or trigger spray systems. The formulations may achieve a low VOC-content, optimal fallout characteristics, and improved spray characteristics. Both water-based and water-free formulations are provided, and they may include a fragrance. In one example, the formulation includes a solvent system with ethanol, acetone, and an ester selected from dimethyl carbonate and methyl acetate and optionally water.

### CROSS REFERENCE TO RELATED APPLICATIONS

This application is related to, claims priority to, and incorporates herein by reference for all purposes U.S. Provisional Patent Application No. 63/522,338, filed Jun. 21, 2023.

### REFERENCE REGARDING FEDERALLY SPONSORED RESEARCH OR DEVELOPMENT

Not applicable.

### SEQUENCE LISTING

Not Applicable.

### BACKGROUND

### 1. Technology Field

The present disclosure relates generally to formulations having a solvent mixture with a low VOC-content, optimal fallout characteristics, and improved spray characteristics.

### 2. Description of the Background

Aerosol and trigger formulations, including air treatment and insecticide compositions, may be water-based formulations that deliver fragrance or active ingredients to the air or ambient environment. Such compositions, however, can have a high volatile organic content (VOC) level, and in some cases, it is desirable to reduce in part or whole the VOC levels of the aerosol, compressed gas, or trigger compositions. In some cases, the volatile solvents of certain compositions have been replaced in part with water to provide a formulation with higher aqueous content and lower VOC levels. These water-based formulations, however, can produce large particle sizes, which leads to higher fallout of the product or formulation and slower evaporation rates when dispensed, which is typically undesirable for volatile compositions.

Therefore, it is continuously desirable to have compositions or formulations, including water-based or aqueous formulations, with a low VOC-content that have improved or lower fallout, as well as quicker evaporation rates and other optimal spray characteristics.

### SUMMARY

Disclosed herein are formulations with a solvent system that may be used in aerosol, compressed gas, or trigger spray systems.

One aspect of the present invention provides a formulation. The formulation comprises between about 10 wt. % to about 40 wt. % of a solvent system, a fragrance, and water. The solvent system comprises ethanol, acetone, and an ester selected from dimethyl carbonate and methyl acetate. All weight percentages are percent by weight of the total formulation.

In some embodiments, the ester comprises methyl acetate. In some embodiments, the ester comprises dimethyl carbonate. In some embodiments, the ethanol is between about 0.5 wt. % to about 10 wt. %, the acetone is between about 5 wt. % to about 20 wt. %, and the ester is between about 5 wt. % to about 20 wt. % of the total formulation. In some embodiments, the ethanol has lower weight percentages than the acetone or the ester in the solvent system. In some embodiments, the ratio of the total amount of non-ethanol solvents to ethanol in the solvent system is at least 4:1. In some embodiments, the fragrance is between about 0.01 wt. % to about 5 wt. % of the total formulation. In some embodiments, the water is between about 60 wt. % to about 99 wt. % of the total formulation. In some embodiments, the formulation further comprises a propellant selected from the group consisting of nitrogen, an inert gas, air, nitrous oxide, carbon dioxide, or mixtures thereof. In some embodiments, the formulation further comprises a co-solvent, an odor active, a surfactant, a pH adjuster, a buffering agent, or any combinations thereof.

Another aspect of the present invention provides a formulation. The formulation comprises between about 80 wt. % to about 99.5 wt. % of a solvent system and a fragrance. The solvent system comprises ethanol, acetone, and an ester selected from dimethyl carbonate and methyl acetate. All weight percentages are percent by weight of the total formulation.

In some embodiments, the ester comprises methyl acetate. In some embodiments, the ester comprises dimethyl carbonate. In some embodiments, the ratio of the total amount of non-ethanol solvents to ethanol in the solvent system is at least 4:1. In some embodiments, the ethanol has lower weight percentages than the total amount of non-ethanol solvents in the solvent system. In some embodiments, the ethanol is between about 5 wt. % to about 20 wt. %, the acetone is between about 5 wt. % to about 20 wt. %, and the ester is between about 70 wt. % to about 95 wt. % of the total formulation. In some embodiments, the ratio of the total amount of non-ethanol solvents to ethanol in the solvent system is at least 8:1. In some embodiments, the fragrance is between about 0.01 wt. % to about 5 wt. % of the total formulation. In some embodiments, the formulation further comprises a propellant, a co-solvent, an odor active, a surfactant, a pH adjuster, a buffering agent, or any combinations thereof.

Another aspect of the present invention provides a formulation. The formulation comprises a solvent system, which comprises between about 1 wt. % to about 15 wt. % of ethanol, between about 5 wt. % to about 15 wt. % of acetone, and between about 5 wt. % to about 95 wt. % of an ester selected from dimethyl carbonate and methyl acetate. All weight percentages are percent by weight of the total formulation.

In some embodiments, the ethanol has lower weight percentages than the total amount of non-ethanol solvents in the solvent system. In some embodiments, the ester is between about 5 wt. % to about 20 wt. % of the total formulation and the ratio of the total amount of non-ethanol solvents to ethanol in the solvent system is at least 4:1. In some embodiments, the ester is between about 70 wt. % to about 95 wt. % of the total formulation and the ratio of the total amount of non-ethanol solvents to ethanol in the solvent system is at least 8:1.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is a graph illustrating a comparison of the percent fallout from various spray formulations; and

FIG. 2 is a graph illustrating the evaporation time of various spray formulations.

### DETAILED DESCRIPTION OF THE INVENTION

The present disclosure provides formulations having solvents that provide the formulation with a low VOC-content, optimal fallout characteristics, and improved spray characteristics. As will be described herein, the characteristics of the solvents disclosed herein impact the spray characteristics. Further, in the specific context of fragrance dispensing systems, fallout is a spray characteristic that results from the aerosol spray, which can be a nuisance by creating residue along various surfaces within a spray zone. More particularly, the unwanted residue that results from increased fallout is generally an undesirable effect that can cause a wetness that is unwanted by consumers or users of the formulations. More so, many prior art dispensing systems or formulations dispense inconsistent sprays over the life of the product and fail to provide sufficient fragrance coverage within an enclosed room or area.

The term “weight percent”, “wt. %”, “wt. %”, “percent by weight”, “% by weight”, and variations thereof, as used herein, refer to the concentration of a substance as the weight of that substance divided by the total weight of the composition or formulation and multiplied by 100. It is understood that, as used here, “percent”, “%”, and the like may be synonymous with “weight percent”, “wt. %”, etc.

As used herein, the term “fragrance” is meant as including any perfume or aroma ingredient or a mixture thereof. A “fragrance” is meant here as a compound which is of current use in the perfumery or aroma industry, i.e., a compound which is used as an active ingredient in perfumed or aroma candles in order to impart a hedonic effect into its ambient or surrounding environment. Put differently, a “fragrance” is an ingredient or mixture that imparts or modifies a surrounding environment with a positive or pleasant odor. More so, this definition is also meant to include compounds that do not necessarily have an odor, but are capable of modulating the odor of a perfuming composition and, as a result, of modifying the perception by a user of the odor of such a composition. In general, these fragrance ingredients belong to chemical classes as varied as alcohols, aldehydes, ketones, esters, ethers, acetates, nitriles, terpene, hydrocarbons, nitrogenous or sulphureous heterocyclic compounds and essential oils, and said perfuming ingredients can be of natural or synthetic origin.

The present disclosure identifies solvent systems and formulation parameters which have been found to decrease and improve fallout from an aerosol or compressed gas dispensing system or a trigger system. Beyond fallout characteristics, other spray characteristics that are considered when evaluating these formulations include evaporation rate, particle size, discharge rate, angle of spray, throw distance, spray cone diameter, percent fallout, fallout pattern, and particle velocity.

Additionally, when referenced herein, “fallout” or “fall out” may generally refer to the residue created by an aerosol, compressed gas, or trigger spray system along various surfaces within a spray zone. Further, the unwanted residue that results from increased fallout is generally an undesirable effect and can cause a wetness that is unwanted by consumers. Fallout can also be characterized as a wetness of the spray plume in the air and/or a build-up of residue on surfaces after use of a dispensing system. The present disclosure identifies key spray characteristics and formulation parameters-including a solvent system-which have been found to decrease and/or improve fallout from an aerosol, compressed gas, or trigger spray system. These formulation parameters and solvent systems have also improved other spray characteristics, such as an evaporation rate of the formulation.

For each formulation, the percent fallout was measured. Percent fallout tests measure the amount of aerosol or spray liquid that falls to the ground after the formulation has been sprayed in the air. In order to conduct this test, the substrate consists of ten sheets of Kraft paper, each 36″×9″. Each are numbered one through ten, individually weighed and placed on the ground in a 36″×90″ array to define a spray surface. Before each product was tested, the product was weighed to determine an initial weight (Wi). The product was then sprayed at a particular height of five feet for five seconds in a direction of the substrate. After the aerosol or spray settled, the weight of the liquid, or fallout, on each sheet (Ws) was recorded and the product was weighed again to determine a final weight (Wf). Using the difference between the initial weight (Wi) and the final weight (Wf) and the total weight of the liquid on the substrates (Ws), a percent fallout was determined using Equation 1 below. After the percent fallout was determined, the substrates were replaced, and the test may be repeated additional times for each product at the same height.

$$Percent Fallout = \frac{{W}_{s}}{{W}_{i} - {W}_{f}} * 100 \% \qquad ( Equation 1 )$$

Evaporation times of the formulations disclosed herein are also measured. In some embodiments, evaporation time may be measured by dispensing a formulation on a mirror using a pump sprayer. The output of the fine mist sprayer is measured to be 0.12 grams per actuation. The finger pump is placed into the sample bottle, primed three times, and then sprayed once onto a 12″×12″ glass mirror from a distance of 12″ at a 45° angle. The time measured where about 90% of the spray has evaporated is denoted as the evaporation time. The test may be done in triplicates to determine statistical differences. Further, the same single pump sprayer is used for all testing for consistency. Subsequent formulation testing involves removing the pump sprayer from the bottle, wiping it clean, and clearing until no additional product is dispensed by continuously pumping the sprayer. The pump is then placed into the next sample bottle, primed three times and the testing is repeated. Additionally, the glass mirrors are washed with water and dried with Kimwipes between each testing.

The solvent system in the formulations described herein may contain one or more VOC-exempt solvents. VOC exempt solvents include organic compounds that are exempt from restrictions placed on volatile organic compounds (VOCs) by the U.S. EPA, such as acetone (CAS #67-64-1), dimethyl carbonate (CAS #616-38-6), methyl acetate (CAS #79-20-9), t-butyl acetate (CAS #540-88-5), propylene carbonate (CAS #108-32-7), and parachlorobenzotrifluoride (Oxsol 100, CAS #98-56-6). The VOC exempt solvents in the formulations described herein may also include LVP (low vapor pressure)-VOC solvents, which is a category of solvents that have a vapor pressure of less than 0.1 millimeters of mercury (at 20° C.); or, if the vapor pressure is unknown, consist of more than 12 carbon atoms or have a melting point higher than 20° C. Suitable LVP-VOC solvents for use in the present invention include, but are not limited to, Isopar M (CAS #64742-47-8), DPMA (CAS #88917-22-0), and Augco (CAS #100-79-8).

In some embodiments, the solvent system is miscible in water. In some such embodiments, the solvent system may comprise a VOC solvent with an evaporation rate >0.3 based on n-butyl acetate, such as an alcohol. Suitable alcohols include, but are not limited to, ethanol and isopropyl alcohol. In some embodiments, the VOC solvent component comprises ethanol. However, in these embodiments, it is typically beneficial to use less alcohol in the solvent system to lower the VOC-content. Thus, in such embodiments, other solvents are used in higher concentrations compared to the VOC solvent component (e.g., ethanol).

In some embodiments, the present disclosure provides a formulation comprising a solvent system that includes a ternary blend of solvents. The ternary blend of solvent may comprise a VOC-exempt solvent that is not an LVP-VOC solvent, a second VOC-exempt solvent, and a third solvent that is a VOC solvent. In some embodiments, the ternary blend of solvents comprises an alcohol, such as ethanol; an ester, such as dimethyl carbonate or methyl acetate; and a ketone, such as acetone. In one particular example, the solvent system includes ethanol, acetone, and methyl acetate. In another particular example, the solvent system includes ethanol, acetone, and dimethyl carbonate.

In some embodiments, the solvent system does not include a third solvent and only uses two solvents. In some embodiments, both solvents are VOC-exempt solvents. In some embodiments, the solvent system comprises a VOC-exempt solvent that is not an LVP-VOC solvent, and a second VOC-exempt solvent. In some embodiments, only one of the two solvents in the solvent system is a VOC-exempt solvent. In one particular example, the solvent system includes ethanol and dimethyl carbonate. In another particular example, the solvent system includes acetone and dimethyl carbonate. In other examples the solvent system includes any one or more of ethanol, acetone, and dimethyl carbonate.

As will be discussed further herein, it was determined that a formulation having a blend of solvents, rather than a single solvent, provides better fallout and spray characteristics. In particular, the solvent blends of the present disclosure provide lower percent fallout and quicker evaporation rates. As such, the formulations of the present disclosure allow a higher percentage of aerosol, compressed gas, or trigger sprays to remain suspended within the air, rather than falling to the ground or remaining idle on surfaces in liquid form. As a result, a consumer can spray less product to produce the desired intensity of the active components within the formulations, such as a fragrance. Thus, the lifespan of the product is also increased.

The total amount of the solvent system may be from about 10 wt. % to about 99.5 wt. %, from about 10 wt. % to about 95 wt. %, from about 10 wt. % to about 90 wt. %, from about 10 wt. % to about 80 wt. %, from about 10 wt. % to about 70 wt. %, from about 10 wt. % to about 60 wt. %, from about 10 wt. % to about 50 wt. %, from about 10 wt. % to about 40 wt. %, from about 15 wt. % to about 40 wt. %, from about 20 wt. % to about 40 wt. %, from about 20 wt. % to about 30 wt. %, from about 30 wt. % to about 99.5 wt. %, from about 40 wt. % to about 99.5 wt. %, from about 50 wt. % to about 99.5 wt. %, from about 60 wt. % to about 99.5 wt. %, from about 70 wt. % to about 99.5 wt. %, or from about 80 wt. % to about 99.5 wt. % of the total formulation. In some embodiments, the solvent system is about 27 wt. % of the total formulation. In some embodiments, the solvent system is about 99 wt. % of the total formulation.

The VOC solvent may be from about 0.5 wt. % to about 20 wt. %, from about 0.5 wt. % to about 15 wt. %, from about 0.5 wt. % to about 10 wt. %, from about 0.5 wt. % to about 5 wt. %, from about 1 wt. % to about 20 wt. %, from about 1 wt. % to about 15 wt. %, from about 1 wt. % to about 10 wt. %, from about 1 wt. % to about 8 wt. %, from about 2 wt. % to about 6 wt. %, from about 3 wt. % to about 5 wt. %, from about 5 wt. % to about 20 wt. %, or from about 5 wt. % to about 15 wt. % of the total formulation. In some embodiments, the VOC solvent comprises ethanol. In some embodiments, the ethanol is about 4 wt. % of the total formulation. In some embodiments, the ethanol is about 10 wt. % of the total formulation.

The VOC-exempt solvent may be from about 1 wt. % to about 99 wt. %, from about 1 wt. % to about 95 wt. %, from about 1 wt. % to about 90 wt. %, from about 1 wt. % to about 80 wt. %, from about 1 wt. % to about 70 wt. %, from about 1 wt. % to about 60 wt. %, from about 1 wt. % to about 50 wt. %, from about 1 wt. % to about 40 wt. %, from about 1 wt. % to about 30 wt. %, from about 1 wt. % to about 25 wt. %, from about 1 wt. % to about 20 wt. %, from about 1 wt. % to about 15 wt. %, from about 5 wt. % to about 99 wt. %, from about 5 wt. % to about 95 wt. %, from about 5 wt. % to about 90 wt. %, from about 5 wt. % to about 80 wt. %, from about 5 wt. % to about 70 wt. %, from about 5 wt. % to about 60 wt. %, from about 5 wt. % to about 50 wt. %, from about 5 wt. % to about 40 wt. %, from about 5 wt. % to about 30 wt. %, from about 5 wt. % to about 25 wt. %, from about 5 wt. % to about 20 wt. %, from about 5 wt. % to about 15 wt. %, from about 50 wt. % to about 99 wt. %, from about 60 wt. % to about 99 wt. %, from about 70 wt. % to about 99 wt. %, from about 70 wt. % to about 95 wt. %, from about 75 wt. % to about 99 wt. %, from about 75 wt. % to about 95 wt. %, from about 75 wt. % to about 90 wt. %, or from about 80 wt. % to about 90 wt. % of the total formulation. In some embodiments, the VOC-exempt solvent comprises acetone. In some such embodiments, the acetone is about 10 wt. % of the total formulation. In some embodiments, the VOC-exempt solvent comprises an ester, such as dimethyl carbonate. In some such embodiments, the dimethyl carbonate is about 13 wt. % of the total formulation. In another embodiment, the dimethyl carbonate is about 79 wt. % of the total formulation. In another embodiment, the dimethyl carbonate is about 89 wt. % of the total formulation. In some embodiments, the VOC-exempt solvent comprises an ester, such as methyl acetate. In some such embodiments, the methyl acetate is about 13 wt. % of the total formulation.

In some embodiments, the VOC solvent component is used in lower weight percentages compared to the VOC-exempt solvent component. For example, in some embodiments, the ethanol has lower weight percentages than the acetone, dimethyl carbonate, or methyl acetate in the solvent system. In some embodiments, the ethanol has lower weight percentage than the total amount of non-ethanol solvents in the solvent system.

In some embodiment, the ratio of the VOC-exempt solvent component to the VOC solvent component is at least 2:1, at least 3:1, at least 4:1, at least 5:1, at least 6:1, at least 7:1, at least 8:1, at least 9:1, or at least 10:1. The ratio is calculated using the weight percentage of each solvent in the total formulation. In some embodiments, the VOC solvent component comprises an alcohol, such as ethanol. In some embodiments, the ratio of the total amount of non-ethanol solvents to ethanol in the solvent system is at least 2:1, at least 3:1, at least 4:1, at least 5:1, at least 6:1, at least 7:1, at least 8:1, at least 9:1, or at least 10:1.

In some embodiments, the blend of solvent in the solvent system disclosed herein achieves synergistic effects, such as faster evaporation and/or less fallout, comparing to the isolated components of the solvent system or alternative solvent combinations.

In some embodiments, the formulation disclosed herein comprises water. The water component may be a solvent carrier and can be deionized water, reverse osmosis water, distilled water, tap water, and/or the like.

Water may be from about 10 wt. % to about 99 wt. %, from about 20 wt. % to about 99 wt. %, from about 30 wt. % to about 99 wt. %, from about 40 wt. % to about 99 wt. %, from about 50 wt. % to about 99 wt. %, from about 60 wt. % to about 99 wt. %, from about 60 wt. % to about 90 wt. %, from about 70 wt. % to about 90 wt. %, or from about 60 wt. % to about 80 wt. % of the total formulation. In some embodiments, water is from about 60 wt. % to about 70 wt. % of the total formulation.

In some embodiments, the formulation disclosed herein is water-free.

In some embodiments, the formulation disclosed herein comprises a fragrance. Suitable fragrances can be a natural or synthetic fragrance, based on a single component or a blend of components. Fragrances are commercially available from various fragrance manufacturers, such as Takasago, International Flavors and Fragrances, Inc., Quest, Firmenich, Givaudan, Symrise, and the like.

The fragrance may be from about 0.01 wt. % to about 10 wt. %, from about 0.01 wt. % to about 8 wt. %, from about 0.01 wt. % to about 5 wt. %, from about 0.5 wt. % to about 5 wt. %, from about 0.5 wt. % to about 3 wt. %, from about 0.5 wt. % to about 2 wt. %, or from about 0.5 wt. % to about 1.5 wt. % of the total formulation. In some embodiments, the fragrance is about 1 wt. % of the total formulation.

In some embodiments, the formulation disclosed herein comprises an odor active. In some embodiments, the odor active comprises triethylene glycol.

The odor active may be from about 0.01 wt. % to about 10 wt. %, from about 0.01 wt. % to about 8 wt. %, from about 0.01 wt. % to about 5 wt. %, from about 0.5 wt. % to about 5 wt. %, from about 0.5 wt. % to about 3 wt. %, from about 0.5 wt. % to about 2 wt. %, or from about 0.5 wt. % to about 1.5 wt. % of the total formulation. In some embodiments, the odor active is about 1 wt. % of the total formulation.

In some embodiments, the formulation disclosed herein comprises a propellant. The propellant may be any suitable conventionally known compressed gas, including, but not limited to, nitrogen, an inert gas, air, nitrous oxide, carbon dioxide, or mixtures thereof. In some embodiments, the propellant comprises nitrogen gas.

The propellant may be from about 0.01 wt. % to about 10 wt. %, from about 0.01 wt. % to about 8 wt. %, from about 0.01 wt. % to about 5 wt. %, from about 0.5 wt. % to about 5 wt. %, from about 0.5 wt. % to about 3 wt. %, from about 0.5 wt. % to about 2 wt. %, or from about 0.5 wt. % to about 1.5 wt. % of the total formulation. In some embodiments, the propellant is about 0.68 wt. % of the total formulation.

In some embodiments, the formulations disclosed herein may comprise a co-solvent in addition to the solvent system described above. Suitable co-solvents include, but are not limited to, glycols and glycol ethers. In some embodiments, the co-solvent comprises propylene glycol.

The co-solvent may be from about 0.01 wt. % to about 10 wt. %, from about 0.05 wt. % to about 8 wt. %, from about 0.1 wt. % to about 8 wt. %, from about 0.1 wt. % to about 5 wt. %, from about 0.1 wt. % to about 4 wt. %, from about 0.1 wt. % to about 2 wt. %, from about 0.1 wt. % to about 1 wt. %, or from about 0.4 wt. % to about 0.6 wt. % of the total formulation. In some embodiments, the co-solvent is about 0.46 wt. % of the total formulation.

In some embodiments, the formulations disclosed herein may comprise a surfactant. The surfactant may be nonionic, cationic, anionic, amphoteric, zwitterionic, or mixtures thereof. The surfactant may be generally selected in view of the dispensing container used. For example, a composition stored and dispensed from a steel or steel alloy-based container may include a nonionic and/or amphoteric surfactant (which are less corrosive) whereas an aluminum or plastic container on the other hand can include those and/or other surfactants. The surfactant component may comprise one or more surfactants.

Suitable nonionic surfactants include, but are not limited to, polyalkoxylated hydrogenated castor oil, including polyethoxylated hydrogenated caster oil such as TAGAT CH60 (60 ethylene oxide (EO) units), TAGAT CH40 (40 EO units); hydrogenated and ethoxylated castor oil blends, e.g. EUMULGIN HPS (40 EO units); secondary alcohol ethoxylates, e.g., TERGITOL brand surfactants such as TERGITOL 15-S-12 and TERGITOL 15-S-7; ethoxylated linear alcohols, e.g., LUTENSOL brand such as LUTENSOL A08 (8 EO units); sorbitan monooleate; polyethylene sorbitan monooleate; polyoxyethylene sorbitan monolaurate; alkyl polyglycosides; polyethyleneoxide/polypropyleneoxide; alkyl phenol ethoxylated carboxylated alcohols; and mixtures thereof. In some embodiments, the surfactant comprises hydrogenated castor Oil 60 Ethoxylate, C15 PEG 7 ethoxylated alcohol, or a combination thereof.

The surfactant may be from about 0.01 wt. % to about 10 wt. %, from about 0.05 wt. % to about 8 wt. %, from about 0.1 wt. % to about 8 wt. %, from about 0.1 wt. % to about 5 wt. %, from about 0.1 wt. % to about 4 wt. %, from about 0.1 wt. % to about 2 wt. %, from about 0.1 wt. % to about 1 wt. %, or from about 0.5 wt. % to about 1 wt. % of the total formulation.

In some embodiments, the formulations disclosed herein may comprise a buffering agent. Suitable buffering agents include, but are not limited to, bicarbonates, such as sodium bicarbonate; phosphates; ammonium hydroxide; THAM-Tris (hydroxymethyl) aminoethane; 2-amino-2-methyl-propane diol; and the like. It is noted that some pH buffering agents, such as phosphates, carbonates, ammonium hydroxide, THAM-Tris (hydroxymethyl) aminoethane, and 2-amino-2-methyl-propane diol, can provide a multi-purpose function of corrosion inhibitor, pH adjustor, and buffering agent. In such instance, one or a combination of ingredients may be used to meet these functions and amounts thereof adjusted accordingly within the scope of the invention. In some embodiments, the buffering agent comprises monosodium phosphate.

The buffering agent may be from about 0.01 wt. % to about 5 wt. %, from about 0.01 wt. % to about 4 wt. %, from about 0.01 wt. % to about 3 wt. %, from about 0.01 wt. % to about 1 wt. %, or from about 0.05 wt. % to about 1 wt. % of the total formulation. In some embodiments, the buffering agent is about 0.20 wt. % of the total formulation.

In some embodiments, the formulations disclosed herein may comprise a pH adjuster. Suitable pH adjusters include, but are not limited to, carbonates, such as sodium carbonate; silicates, such as sodium meta-silicate pentahydrate; phosphates, such as disodium phosphate, and dipotassium phosphate; hydroxides, such as sodium hydroxide; ammonium hydroxide; THAM-Tris-(hydroxymethyl) aminoethane; 2-amino-2-methyl-propane diol; and the like. Some of these compounds may also provide a dual function as a pH adjuster and corrosion inhibitor. In some embodiments, the pH adjuster comprises sodium carbonate.

The pH adjuster may be from about 0.01 wt. % to about 5 wt. %, from about 0.01 wt. % to about 4 wt. %, from about 0.01 wt. % to about 3 wt. %, from about 0.01 wt. % to about 1 wt. %, from about 0.05 wt. % to about 1 wt. %, from about 0.05 wt. % to about 0.5 wt. %, or from about 0.05 wt. % to about 0.2 wt. % of the total formulation. In some embodiments, the pH adjuster is about 0.18 wt. % of the total formulation.

Any of the embodiments described herein may be modified to include any of the structures, compositions, or methodologies disclosed in connection with different embodiments.

In some embodiments, the numbers expressing quantities of ingredients, properties such as weight percentages, fallout percentages, evaporation rates, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the present disclosure (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### EXAMPLES

### Example 1

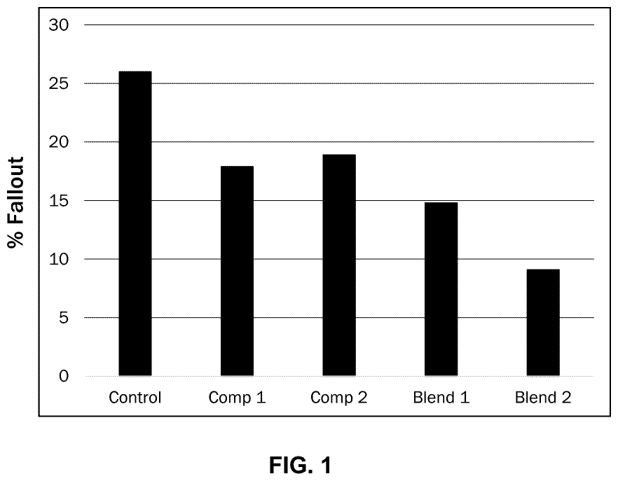

FIG. 1 is a graph illustrating a comparison of the percent fallout from various spray formulations at a height of 5 feet. The data of FIG. 1 is further shown in Table 2 below, which illustrates experimental results from a percent (%) fallout test conducted with a compressed gas system having the solvent systems of the present disclosure, as well as several comparable dispensing systems and a control system. The formulations used to conduct the test are shown in Table 1 below. With reference to Table 1 below, a control formulation was tested with the blends of the present disclosure. Further, two comparable formulations, including comparable formulation 1 and comparable formulation 2, were tested. Blend 1 and Blend 2 both incorporate solvent systems of the present disclosure. The values shown under each formulation represent the weight percentages (wt. %) of components.

TABLE 1

| | | Comp | Comp | Blend | Blend |
|---|---|---|---|---|---|
| Composition | Control | 1 | 2 | 1 | 2 |
| Purified Water | qs | qs | qs | qs | qs |
| Ethanol | 9 | 30 | — | 4 | 4 |
| Acetone | — | — | — | 10 | 10 |
| Dimethyl Carbonate | — | — | 13 | 13 | — |
| Methyl Acetate | — | — | — | — | 13 |
| Fragrance | 1 | 1 | 1 | 1 | 1 |
| Triethylene Glycol | 1 | 1 | 1 | 1 | 1 |
| Nitrogen Gas | 0.68 | 0.68 | 0.68 | 0.68 | 0.68 |
| Hydrogenated Castor | 0.47 | 0.47 | 0.47 | 0.47 | 0.47 |
| Oil 60 Ethoxylate | | | | | |
| Propylene Glycol | 0.46 | 0.46 | 0.46 | 0.46 | 0.46 |
| C15 PEG 7 | 0.28 | 0.28 | 0.28 | 0.28 | 0.28 |
| ethoxylated alcohol | | | | | |
| Monosodium | 0.20 | 0.20 | 0.20 | 0.20 | 0.20 |
| phosphate | | | | | |
| Sodium Carbonate | 0.18 | 0.18 | 0.18 | 0.18 | 0.18 |

The formulations of Table 1 could also be prepared without nitrogen gas, for instance for use as a trigger spray solution.

For each formulation, the percent fallout was measured. Percent fallout tests measure the amount of aerosol or spray liquid that falls to the ground after the formulation has been sprayed in the air. In order to conduct this test, the substrate consists of ten sheets of Kraft paper, each 36″×9″. Each are numbered one through ten, individually weighed and placed on the ground in a 36″×90″ array to define a spray surface. Before each product was tested, the product was weighed to determine an initial weight (Wi). The product was then sprayed at a particular height of five feet for five seconds in a direction of the substrate. After the aerosol or spray settled, the weight of the liquid, or fallout, on each sheet (Ws) was recorded and the product was weighed again to determine a final weight (Wf). Using the difference between the initial weight (Wi) and the final weight (Wf) and the total weight of the liquid on the substrates (Ws), a percent fallout was determined using Equation 1 below. After the percent fallout was determined, the substrates were replaced, and the test may be repeated additional times for each product at the same height.

$$Percent Fallout = \frac{{W}_{s}}{{W}_{i} - {W}_{f}} * 100 \% \qquad ( Equation 1 )$$

TABLE 2

| | Spray | Spray | Percent |
|---|---|---|---|
| Formulation | Height (ft) | Duration (s) | Fallout |
| Control | 5 | 5 | about 26 |
| Comp 1 | 5 | 5 | about 18 |
| Comp 2 | 5 | 5 | about 19 |
| Blend 1 | 5 | 5 | about 14.5 |
| Blend 2 | 5 | 5 | about 9 |

As illustrated in FIG. 1 and Table 2, the solvent systems of the present disclosure, which are included in Blend 1 and Blend 2, produced the lowest percent fallout versus the other formulations. In some instances, the percent fallout for the formulations of the present disclosure was almost half the comparable formulations. Therefore, the formulations of the present disclosure allow a higher percentage of aerosol, compressed gas, or trigger sprays to remain suspended within the air rather than falling to the ground. As a result, a consumer can spray less product to produce the desire intensity of the fragrance, which increases the life span of the product. In some embodiments, the formulations of the present disclosure produce a percent fallout at five feet between about 9% and about 20%, or between about 9% and about 15%, or between about 9% and about 14.5%. Further, the percent fallout for the formulations of the present disclosure at five feet may be about 9% or about 14.5%.

### Example 2

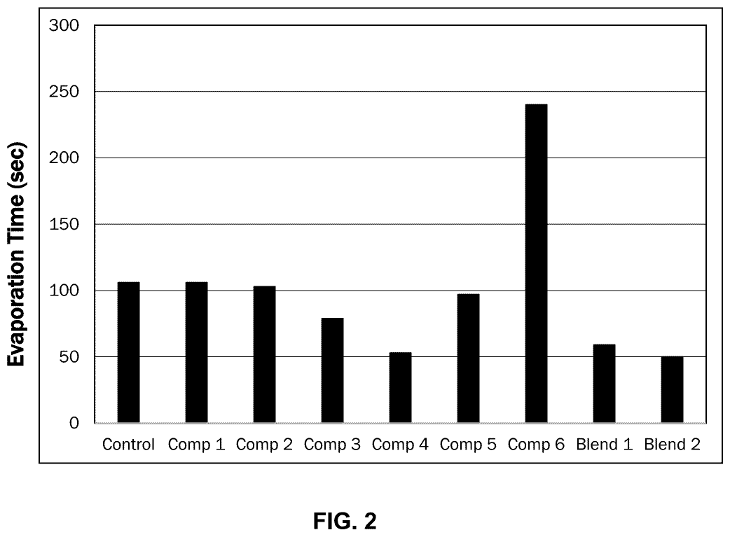

FIG. 2 is a graph illustrating a comparison of the evaporation time of various spray formulations. In this example, each spray formulation is dispensed on a mirror using a pump spray and then a time of evaporation was measured. More particularly, the solvent blends were prepared in a 4 oz. amber glass bottle with a finger pump sprayer. The output of the fine mist sprayer was measured to be 0.12 grams per actuation. The finger pump was placed into the sample bottle, primed three times, and then sprayed once onto a 12″×12″ glass mirror from a distance of 12″ at a 45° angle. The time measured where about 90% of the spray has evaporated is denoted as the evaporation time. The test was done in triplicates to determine statistical differences. Further, the same single pump sprayer was used for all testing for consistency. Subsequent formulation testing required removing the pump sprayer from the bottle, wiping it clean, and clearing until no additional product was dispensed by continuously pumping the sprayer. The pump was then placed into the next sample bottle, primed three times and the testing was repeated. Additionally, the glass mirrors were washed with water and dried with Kimwipes between each testing.

The data of FIG. 2 is further shown in Table 5 below, which illustrates experimental results for the evaporation rate of several formulations. Further, the formulations used to conduct the test are shown in Tables 3 and 4 below. With reference to Tables 3 and 4, a control formulation, six comparable formulations, and five blend formulations that utilize the solvent system of the present disclosure were tested. The values shown under each formulation represent the weight percentages (wt. %) of components.

TABLE 3

| | Con- | Comp | Comp | Comp | Comp | Comp |
|---|---|---|---|---|---|---|
| Composition | trol | 1 | 2 | 3 | 4 | 5 |
| Purified Water | 98 | — | — | — | — | — |
| (with surfactants) | | | | | | |
| Purified Water | — | 100 | 69 | 69 | 69 | 86 |
| Ethanol | — | — | 30 | — | — | — |
| Acetone | — | — | — | 30 | — | — |
| Methyl Acetate | — | — | — | — | 30 | — |
| Dimethyl | — | — | — | — | — | 13 |
| Carbonate | | | | | | |
| Propylene | — | — | — | — | — | — |
| Carbonate | | | | | | |
| Fragrance | 1 | — | 1 | 1 | 1 | 1 |

TABLE 4

| | Comp | Blend | Blend | Blend | Blend | Blend |
|---|---|---|---|---|---|---|
| Composition | 6 | 1 | 2 | 3 | 4 | 5 |
| Purified Water (with | — | — | — | | | |
| surfactants) | | | | | | |
| Purified Water | 69 | qs | qs | | 10 | 10 |
| Ethanol | — | 4 | 4 | 10 | | 10 |
| Acetone | — | 10 | 10 | | | |
| Methyl Acetate | — | 13 | — | | | |
| Dimethyl Carbonate | — | — | 13 | 89 | 89 | 79 |
| Propylene Carbonate | 30 | — | — | | | |
| Fragrance | 1 | 1 | 1 | 1 | 1 | 1 |

TABLE 5

| | Evaporation |
|---|---|
| Composition | Rate(s) |
| Control | 106 |
| Comp 1 | 106 |
| Comp 2 | 103 |
| Comp 3 | 79 |
| Comp 4 | 53 |
| Comp 5 | 97 |
| Comp 6 | 240 |
| Blend 1 | 59 |
| Blend 2 | 50 |
| Blend 3 | 5 |
| Blend 4 | 5 |
| Blend 5 | 5 |

As illustrated in FIG. 2 and Table 5, the solvent systems of the present disclosure produced the quickest evaporation rates versus other tested formulations. In some embodiments, the evaporation rates of the formulations of the present disclosure were almost half, or significantly less than the evaporation rates of the comparable formulations. Therefore, the formulations of the present disclosure allow a higher percentage of active components of aerosol, compressed gas, or trigger systems to remain suspended in the air, rather than remaining idle on a surface. As a result, a consumer can spray less product to produce the desired intensity of the actives therein, such as a fragrance. These improved evaporation rates also increase the lifespan of the product.

### Example 3

The evaporation rate of solvents and several components of the formulations were also tested in isolation and compared to the solvent systems of the present disclosure. In particular, the evaporation rate of propylene carbonate, methyl acetate, dimethyl carbonate, acetone, TB acetate, ethanol, and water were tested and compared to a solvent system of the present disclosure that includes ethanol, acetone, dimethyl carbonate, and water. The evaporation rate was measured using the same procedures discussed herein in connection with Example 2. Table 6 provides the results of this test.

TABLE 6

| | Evaporation | Rank |
|---|---|---|
| Composition | Rate(s) | Evaporation |
| Propylene Carbonate (30%) | 240 | 8 |
| Methyl acetate (30%) | 53 | 1 |
| Dimethyl Carbonate (13%) | 97 | 4 |
| Acetone (30%) | 79 | 3 |
| TB Acetate (30%) | 103 | 5 |
| Ethanol (30%) | 103 | 5 |
| Water only | 106 | 7 |
| 4% Ethanol, 10% acetone, | 59 | 2 |
| 13% carbonate, 73% water | | |

As shown in Table 6, the solvent system of the present disclosure had improved evaporation rates compared to other solvents. Additionally, Table 6 illustrates the synergistic effects of the solvent blends. In particular, the solvent blends of the present disclosure had quicker evaporation compared to the isolated components of the solvent blend.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

It will be appreciated by those skilled in the art that while the invention has been described above in connection with particular embodiments and examples, the invention is not necessarily so limited, and that numerous other embodiments, examples, uses, modifications and departures from the embodiments, examples, and uses are intended to be encompassed by the claims attached hereto. The entire disclosure of each patent and publication cited herein is incorporated by reference, as if each such patent or publication were individually incorporated by reference herein.

Any of the embodiments described herein may be modified to include any of the structures or methodologies disclosed in connection with different embodiments. Further, the present disclosure is not limited to any specific aerosol container, compressed gas container, or trigger spray system. Rather, the formulations of any of the embodiments disclosed herein may be modified to work with any type of aerosol or non-aerosol container.

### INDUSTRIAL APPLICABILITY

Numerous modifications to the present invention will be apparent to those skilled in the art in view of the foregoing description. Accordingly, this description is to be construed as illustrative only and is presented for the purpose of enabling those skilled in the art to make and use the invention. The exclusive rights to all modifications which come within the scope of the appended claims are reserved.

We claim:

1. A formulation, comprising:
between about 10 wt. % to about 40 wt. % of a solvent system, the solvent system comprising ethanol, acetone, and an ester selected from dimethyl carbonate and methyl acetate;
a fragrance; and
water,
wherein all weight percentages are percent by weight of the total formulation.

2. The formulation of claim 1, wherein the ester comprises methyl acetate.

3. The formulation of claim 1, wherein the ester comprises dimethyl carbonate.

4. The formulation of claim 1, wherein the ethanol is between about 0.5 wt. % to about 10 wt. %, the acetone is between about 5 wt. % to about 20 wt. %, and the ester is between about 5 wt. % to about 20 wt. %.

5. The formulation of claim 1, wherein the ethanol has lower weight percentage than the acetone or the ester in the solvent system.

6. The formulation of claim 1, wherein the ratio of the total amount of non-ethanol solvents to ethanol in the solvent system is at least 4:1.

7. The formulation of claim 1, wherein the fragrance is between about 0.01 wt. % to about 5 wt. %.

8. The formulation of claim 1, wherein the water is between about 60 wt. % to about 99 wt. %.

9. The formulation of claim 1, further comprising a propellant selected from the group consisting of nitrogen, an inert gas, air, nitrous oxide, carbon dioxide, or mixtures thereof.

10. The formulation of claim 1, further comprising a co-solvent, an odor active, a surfactant, a pH adjuster, a buffering agent, or any combinations thereof.

11. A formulation, comprising:
between about 80 wt. % to about 99.5 wt. % of a solvent system, the solvent system comprising ethanol, acetone, and an ester selected from dimethyl carbonate and methyl acetate; and
a fragrance;
wherein all weight percentages are percent by weight of the total formulation.

12. The formulation of claim 11, wherein the ester comprises methyl acetate.

13. The formulation of claim 11, wherein the ester comprises dimethyl carbonate.

14. The formulation of claim 11, wherein the ethanol has lower weight percentage than the total amount of non-ethanol solvents in the solvent system.

15. The formulation of claim 11, wherein the ethanol is between about 5 wt. % to about 20 wt. %, the acetone is between about 5 wt. % to about 20 wt. %, and the ester is between about 70 wt. % to about 95 wt. %.

16. The formulation of claim 11, wherein the ratio of the total amount of non-ethanol solvents to ethanol in the solvent system is at least 8:1.

17. The formulation of claim 11, wherein the fragrance is between about 0.01 wt. % to about 5 wt. %.

18. The formulation of claim 11, further comprising a propellant, a co-solvent, an odor active, a surfactant, a pH adjuster, a buffering agent, or any combinations thereof.

19. A formulation that comprises a solvent system, the solvent system comprising:
between about 1 wt. % to about 15 wt. % of ethanol,
between about 5 wt. % to about 15 wt. % of acetone, and
between about 5 wt. % to about 95 wt. % of an ester selected from dimethyl carbonate and methyl acetate;
wherein all weight percentages are percent by weight of the total formulation.

20. The formulation of claim 19, wherein the ethanol has lower weight percentage than the total amount of non-ethanol solvents in the solvent system.

21. The formulation of claim 19, wherein the ester is between about 5 wt. % to about 20 wt. % and the ratio of the total amount of non-ethanol solvents to ethanol in the solvent system is at least 4:1.

22. The formulation of claim 19, wherein the ester is between about 70 wt. % to about 95 wt. % and the ratio of the total amount of non-ethanol solvents to ethanol in the solvent system is at least 8:1.
